(19) United States
(12) Patent Application Publication
(10) Pub. No.: US 2022/0395009 A1
(43) Pub. Date: Dec. 15, 2022

(54) ENTERAL SUSTAINED-RELEASE SUGAR ALCOHOL ADDITIVE, PREPARATION METHOD THEREFOR AND APPLICATION THEREOF

(71) Applicant: Zhejiang Huakang Pharmaceutical Co., Ltd., Quzhou, Zhejiang (CN)

(72) Inventors: Shasha XIANG, Quzhou, Zhejiang (CN); Xuan ZHU, Quzhou, Zhejiang (CN); Lihua SHI, Quzhou, Zhejiang (CN); Kun YE, Quzhou, Zhejiang (CN); Mian LI, Santa Clara, CA (US)

(21) Appl. No.: 17/775,193

(22) PCT Filed: Feb. 7, 2021
(86) PCT No.: PCT/CN2021/075696

§ 371 Date: May 6, 2022

(30) Foreign Application Priority Data
Feb. 11, 2020 (CN) 202010087312.6

(51) Int. Cl. A23L 29/30; A23L 29/256; A23L 33/15; A23L 33/125; A23L 33/135; A23P 20/15; A61K 47/36; A61K 31/047; A61K 31/7004; A61K 31/714; A61K 35/747

(52) U.S. Cl. CPC A23L 29/37; A23L 29/256; A23L 33/15; A23L 33/125; A23L 33/135; A23P 20/15; A61K 47/36; A61K 31/047; A61K 31/7004; A61K 31/714; A61K 35/747; A23V 2002/00

(57) ABSTRACT

The present invention relates to an enteral sustained-release sugar alcohol additive including an inner layer structure and an outer layer structure. The inner layer structure contains components of xylitol and agar, and the outer layer structure contains components of carrageenan or gellan gum or xanthan gum or guar gum, vitamin B12, L-arabinose, and fermented bifidobacterium or fermented propionibacterium or fermented lactobacillus. The present invention further provides a method of preparing an enteral sustained-release sugar alcohol additive and an application thereof. In the present invention, by compounding xylitol, L-arabinose and vitamin B12, directional regulation of intestinal microorganisms can be achieved and high concentration synthesis of propionate in the intestine can be realized.

### TECHNICAL FIELD

The present invention relates to the field of preparation technologies of enteral sustained-release products, in particular to an enteral sustained-release sugar alcohol additive and a preparation method and an application thereof.

### BACKGROUND

After being absorbed, propionate is mainly metabolized in liver to participate in gluconeogenesis and inhibit synthesis of cholesterol. However, various researches show that propionates ingested exogenously and generated in body have different effects. The exogenous propionate has significant impact on many metabolic processes of the individuals. Propionate is a short-chain fatty acid. Under normal circumstances, microorganisms in a human body are capable of generating propionate by fermenting un-completely digested carbohydrate in colon and the propionate is beneficial to the human body. But, the exogenous propionate may lead to hyperglycemia and a high level of insulin in a short period, and may even lead to more severe symptoms such as obesity and insulin resistance or the like after a long period of ingestion.

Fructo-oligosaccharide and polysaccharide have the function of generating propionate in intestinal tract. But, neither of fructo-oligosaccharide and polysaccharide can synthesize propionate with a high concentration at a fixed point. The enteral utilization rate of polysaccharide is low and may easily lead to problems of flatulence and the like. Fructo-oligosaccharide may also lead to the problems of excess generation of gas and unstable generation of acid and the like, and even to metabolic disorder of liver and the like due to excess generation of fructose.

### SUMMARY

In order to solve the above technical problems, the present invention provides an enteral sustained-release sugar alcohol additive and a preparation method and an application thereof so as to promote human body health and eliminate metabolic side effect possibly resulting from propionate, thereby achieving slow release of the sugar alcohol additive in intestinal tract and its fixed-point release in colon.

The present invention is achieved by providing an enteral sustained-release sugar alcohol additive. The enteral sustained-release sugar alcohol additive includes an inner layer structure and an outer layer structure, wherein the inner layer structure contains components of xylitol and agar, and the outer layer structure contains components of carrageenan or gellan gum or xanthan gum or guar gum, vitamin B12, L-arabinose, and fermented bifidobacterium or fermented propionibacterium or fermented lactobacillus.

The present invention is achieved by further providing a method of preparing the enteral sustained-release sugar alcohol additive as described above. The method includes the following steps:

at step 1, mixing and sterilizing xylitol and agar, placing the mixture into a ball mould and standing the mixture for cooling so as to prepare xylitol agar pellets;

at step 2, preparing a carrageenan solution or gellan gum solution or xanthan gum solution or guar gum solution, adding vitamin B12 and L-arabinose to the carrageenan solution or gellan gum solution or xanthan gum solution or guar gum solution and mixing to uniformity so as to obtain a mixed solution, and then wrapping the xylitol agar pellets in step 1 with the mixed solution to obtain xylitol agar two-layer colloidal pellets wrapped with the mixed solution;

at step 3, placing the two-layer colloidal pellets prepared in step 2 into a fermented bifidobacterium medium, or a fermented propionibacterium medium or a fermented lactobacillus medium for culturing, and then taking out the pellets for freeze or hot air drying to obtain a finished sugar alcohol additive.

The present invention is achieved by further providing an application of the enteral sustained-release sugar alcohol additive as described above. The sugar alcohol additive is applied to foodstuff.

The present invention is achieved by further providing an application of an enteral sustained-release sugar alcohol additive prepared using the above method of preparing an enteral sustained-release sugar alcohol additive. The sugar alcohol additive is applied to foodstuff.

Compared with the prior art, the enteral sustained-release sugar alcohol additive and the preparation method and the application thereof in the present invention have the following advantages.

1. With xylitol, L-arabinose and vitamin B12, directional regulation to intestinal microorganisms is achieved and enteral high-concentration synthesis of propionate can be realized.

2. With double layer embedding technology, enteral sustained release and layer-by-layer release of sugar alcohol can be achieved.

3. With the pellet technology of the method, stability of bifidobacterium or propionibacterium or lactobacillus products is achieved.

### BRIEF DESCRIPTION OF THE DRAWINGS

FIG. 1 is a schematic diagram illustrating a change of a content of xylitol in an enteral sustained-release sugar alcohol additive in a simulation digestion process of stomach, small intestine and colon according to the present invention.

FIG. 2 is a schematic diagram illustrating a change of a content of L-arabinose in an enteral sustained-release sugar alcohol additive in a simulation digestion process of stomach, small intestine and colon according to the present invention.

FIG. 3 is a comparative schematic diagram of relative abundances of bifidobacterium of a two-layer colloidal pellet product and a finished enteral sustained-release sugar alcohol additive in a simulation colon experiment according to the present invention.

### DETAILED DESCRIPTION OF THE EMBODIMENTS

In order to make the technical problems, technical solutions and beneficial effects of the present invention clearer, the present invention will be further described below in combination with the accompanying drawings and the specific embodiments. It should be understood that the specific embodiments described herein are merely used to explain the present invention rather than limit the present invention.

An enteral sustained-release sugar alcohol additive according to the present invention includes an inner layer structure and an outer layer structure, where the inner layer structure contains components of xylitol and agar, and the outer layer structure contains components of carrageenan or gellan gum or xanthan gum or guar gum, vitamin B12, L-arabinose, and fermented bifidobacterium or fermented propionibacterium or fermented lactobacillus.

Specifically, in the sugar alcohol additive, a component ratio of xylitol to L-arabinose to vitamin B12 is 1-33:1:0.005.

In the enteral sustained-release sugar alcohol additive of the present invention, by compounding xylitol, L-arabinose and vitamin B12, L-arabinose slows down metabolism of xylulose to arabitol and controls metabolic flux, vitamin B12 increases propionate metabolism pathways. In this way, increased gene expressions of enterobacterium and lachnospiraceae relating to xylitol are induced firstly, thereby increasing metabolism of xylitol to PPP pathway, weakening the pathway in which xylulose is metabolized to arabitol and increasing propionate synthesis amount.

In the present invention, the enteral sustained-release sugar alcohol additive adopts two layers of structures. The two-layer colloid prepared using hydrocolloid such as carrageenan and L-arabinose and vitamin B12 can realize sustained release of xylitol and L-arabinose in intestinal tract and their fixed-point release in colon.

The present invention further provides a method of preparing the enteral sustained-release sugar alcohol additive as described above. The method includes the following steps.

At step 1, xylitol and agar are mixed and sterilized and then placed into a ball mould for standing cooling, so as to prepare xylitol agar pellets.

At step 2, a carrageenan solution or gellan gum solution or xanthan gum solution or guar gum solution is prepared, vitamin B12 and L-arabinose are added to the carrageenan solution or gellan gum solution or xanthan gum solution or guar gum solution and then mixed to uniformity so as to obtain a mixed solution, and then the xylitol agar pellets in step 1 are wrapped with the mixed solution to obtain xylitol agar two-layer colloidal pellets wrapped with the mixed solution.

At step 3, the two-layer colloidal pellets prepared in step 2 are placed into a fermented bifidobacterium medium, or a fermented propionibacterium medium or a fermented lactobacillus medium for culturing, and then taken out for freeze or hot air drying to obtain a finished sugar alcohol additive.

The method of preparing an enteral sustained-release sugar alcohol additive according to the present invention will be further described below in combination with specific embodiments.

### Embodiment 1

According to a first embodiment of the present invention, the method of preparing an enteral sustained-release sugar alcohol additive may include the following steps.

At step 1, a given amount of xylitol was weighed and mixed with 2% agar, and then sterilized for 15 min at a temperature of 121° C.; when the mixture was cooled down to 55° C., the mixture was transferred to a ball mould by sucking using a disposable aseptic rubber head dropper and then stood and cooled down so as to prepare xylitol agar pellets.

At step 2, 2 g of carrageenan was weighed and dissolved in 100 ml of distilled water and then sterilized for 15 min at a temperature of 121° C. and then cooled down to 50° C.-60° C. with the temperature no greater than 60° C. to help vitamin B12 to stabilize and avoid light, and aseptic L-arabinose and vitamin B12 were added at a formulation ratio of 1:0.005 to the carrageenan solution and then mixed to uniformity; then the xylitol agar pellets were wrapped using the above mixed solution in an aseptic super-clean table to prepare two-layer colloidal pellets with a formulation ratio of 1-33:1:0.005.

At step 3, the two-layer colloidal pellets prepared at step 2 were added to a bifidobacterium fermentation (10⁹CFU/mL) broth at a ratio of 1:3 of total volume and stood for culture of 24 hours; then the pellets with bacteria were taken out and then subjected to freeze drying for 1 h-24 h or hot air drying for 30 min so as to obtain a finished sugar alcohol additive.

A new embodiment may be obtained by replacing bifidobacterium with propionibacterium or lactobacillus in the above embodiment.

The enteral sustained-release sugar alcohol additive prepared in the embodiment is selected to perform simulation digestion experiment of stomach, small intestine and colon for verification in the following method.

1) Preparation of simulation gastric fluid: 0.62 g of NaCl, 0.22 g of KCl, 0.05 g of CaCl₂), and 0.12 g of NaHCO₃ were dissolved in 200 ml of distilled water, and adjusted to pH 2.0 using 0.1 M HCl solution to prepare a gastric fluid medium. Then, 1.0 mL of CH₃COONa(1.0 mol/L, pH5), 23.6 g of pepsase and 25.0 g of gastric lipase were added to 100 ml of the gastric medium and then the pH value of the gastric medium was adjusted to 2.0 using 0.1 M HCl solution.

2) Simulation of gastric fluid digestion: based on a dose of 1%-5% of xylitol, the finished sugar alcohol additive pellets of the embodiment 1 were mixed with the simulation gastric fluid at a volume ratio of 1:1 and then subjected to water bath for 0.5 h, 1 h, 2 h, 4 h and 6 h at a temperature of 37° C. to respectively obtain digested samples.

3) Preparation of simulation small intestine digestive fluid: 0.54 g of NaCl, 0.065 g of KCl and 0.033 g of CaCl₂) were dissolved in 100 ml of distilled water and adjusted to pH 7.0 using 0.1 M NaOH solution to prepare a small intestine medium. Then, 6.5 mg of trypsin, 200.0 g of bile salt (4%, w/w) and 50.0 g of trypsin solution (7%, w/w) were added to the small intestine medium respectively, and then stirred continuously at room temperature using 0.1 M NaOH solution to adjust the solution pH to 7.5 so as to simulate a small intestine digestive fluid.

4) Simulation of small intestine digestion: the samples after simulation of gastric fluid digestion were mixed with the small intestine fluid respectively at a volume ratio of 3:7 and then subjected to water bath for 0.5 h, 1 h, 2 h, 4 h, and 6 h at a temperature of 37° C. so as to obtain digested samples respectively.

5) Based on a dose of 1%-5% of xylitol, the two-layer colloidal pellet samples (without bifidobacterium embedded) and the finished sugar alcohol additive pellet samples (with bifidobacterium embedded) in the embodiment 1 were supplemented to CDMN (Chang Dao Mo Ni) colon in vitro simulation system to collect pellet samples simulating colon digestion of 24 h and 48 h.

6) With HPLC, contents of xylitol and arabinose and an abundance value of bifidobacterium retained in the pellets after simulation of gastric digestion, small intestine digestion and colon digestion were measured respectively to obtain comparison curves of FIGS. 1-3.

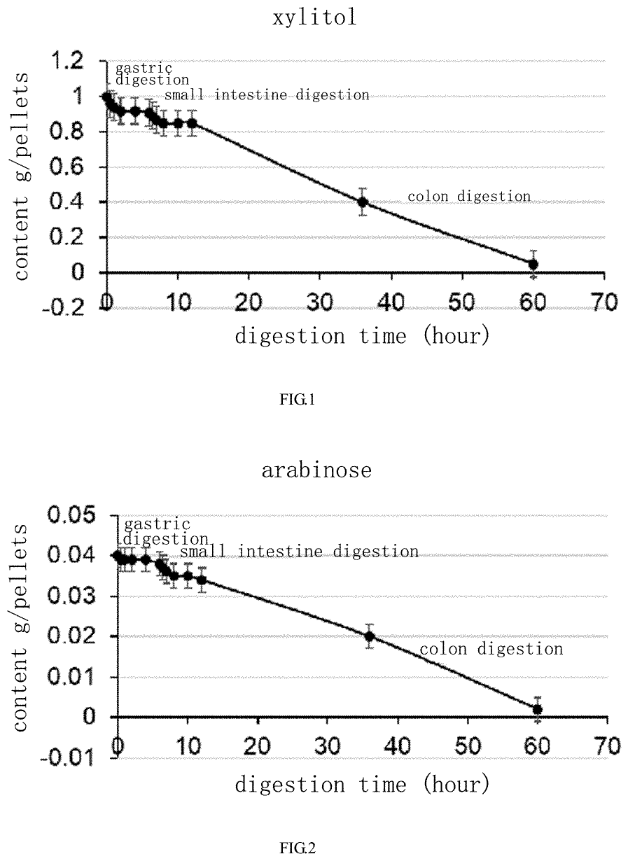

As shown in FIGS. 1 and 2, with xylitol: arabinose=25:1 as an example, in each pellet, the content of xylitol is 1 g, and the content of arabinose is 0.04 g. The first 6 hours relate to gastric digestion, the 6-12 hours relate to digestion of small intestine, and the 12-60 hours relate to contents of xylitol and arabinose retained in the pellets after microbial digestion of large intestine. Compared with the existing experimental data (the absorption rate of the small intestine for xylitol is 60% and the absorption rate for arabinose is 70%), the absorption rate of xylitol is only 15% and the absorption rate of arabinose is only 25% in the pellets of the present invention, and their cumulative release rates in the colon are about 95% respectively. It indicates that the pellets of the present invention have the effect of slow and fixed-point release for xylitol and arabinose.

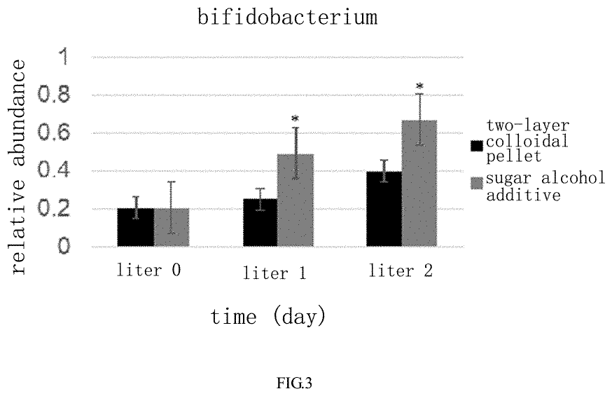

As shown in FIG. 3, compared with non-embedding (two-layer colloidal pellet product), the embedding of bifidobacterium (finished sugar alcohol additive) in the pellets of the present invention can obviously promote the relative abundance of bifidobacterium in the pellets, thereby promoting the relative abundance of bifidobacterium in the colon.

As a result, the enteral sustained-release sugar alcohol additive of the present invention adopts layer-by-layer embedding technology to realize slow and layer-by-layer release of xylitol in intestinal tract and promote the relative abundance of bifidobacterium in the colon.

The present invention further provides an application of the enteral sustained-release sugar alcohol additive as described above. The sugar alcohol additive is applied to foodstuff.

The present invention further provides an application of an enteral sustained-release sugar alcohol additive prepared using the method of preparing an enteral sustained-release sugar alcohol additive as described above. The sugar alcohol additive is applied to foodstuff.

For example, the enteral sustained-release sugar alcohol additive of the present invention is applied to beverage and cakes to promote generation of propionate in the colon of the human body and adjust intestinal health.

The above descriptions are merely made to preferred embodiments of the present invention and shall not be intended to limit the present invention. Any changes, equivalent substitutions and improvements and the like made within the spirit and principle of the present invention shall all fall within the scope of protection of the present invention.

### INDUSTRIAL APPLICABILITY

Insert description paragraphs of the industrial applicability herein. Free text of sequence table

Insert description paragraphs of free text of sequence table herein.

1. An enteral sustained-release sugar alcohol additive, comprising an inner layer structure and an outer layer structure,
wherein the inner layer structure comprises xylitol and agar, and the outer layer structure comprises:
a component comprising carrageenan, gellan gum, xanthan gum, guar gum,
vitamin B12,
L-arabinose, and
a fermented bacterium comprising fermented bifidobacterium, fermented propionibacterium, or fermented lactobacillus.

2. The enteral sustained-release sugar alcohol additive of claim 1, wherein the weight ratio of xylitol to L-arabinose to vitamin B12 is 1-33:1:0.005.

3. A method of preparing the enteral sustained-release sugar alcohol additive according to claim 1, comprising the following steps:
at step 1, mixing and sterilizing xylitol and an agar product to form a mixture, placing the mixture into a ball mould and allowing the mixture for cooling so as to prepare xylitol agar pellets;
at step 2, preparing a solution comprising carrageenan, gellan gum, xanthan gum, or guar gum, adding vitamin B12 and L-arabinose to the solution to obtain a mixed solution, and then coating the xylitol agar pellets in step 1 with the mixed solution to obtain two-layer colloidal pellets;
at step 3, placing the two-layer colloidal pellets prepared in step 2 into a medium comprising fermented bifidobacterium, fermented propionibacterium, or fermented lactobacillus for culturing, and then drying the pellets to obtain the sugar alcohol additive.

4. The method of claim 3, wherein in step 1, the agar product comprises 2% agar, and the mixture comprising xylitol and agar is sterilized for 15 minutes at a temperature of 121° C. before the mixture is placed in the ball mould.

5. The method of claim 3, wherein in step 2, the method of preparing the solution comprising carrageenan, gellan gum, xanthan gum, or guar gum includes: dissolving 2 g of carrageenan or gellan gum or xanthan gum or guar gum in 100 ml of distilled water to form the solution, sterilizing the solution for 15 minutes at a temperature of 121° C., and cooling the solution down to a temperature of 50° C.-60° C.

6. The method of claim 3, wherein in step 3, a concentration of bifidobacterium or propionibacterium or lactobacillus in the medium is 109 CFU/mL, the two-layer colloidal pellets are placed in the medium for culturing for 24 hours, wherein the drying is performed by freeze drying for 1-24 hours or hot air drying for 30 minutes.

7. A foodstuff comprising the sugar alcohol additive of claim 1.

8. A method of preparing a food product, comprising including the sugar alcohol additive prepared from the method of claim 3 into a foodstuff.
